(19) United States
(12) Patent Application Publication
(10) Pub. No.: US 2023/0210076 A1
(43) Pub. Date: Jul. 6, 2023

(54) MODIFIED SEEDS AND PLANTS WITH RESISTANCE TO ENVIRONMENTAL STRESS AND METHODS OF USING THE SAME

(71) Applicant: Texas Tech University System, Lubbock, TX (US)

(72) Inventor: Hong Zhang, Lubbock, TX (US)

(73) Assignee: Texas Tech University System, Lubbock, TX (US)

(21) Appl. No.: 18/085,024

(22) Filed: Dec. 20, 2022

Related U.S. Application Data
(60) Provisional application No. 63/293,175, filed on Dec. 23, 2021.

(51) Int. Cl. A01H 5/10; A01H 6/46

(52) U.S. Cl. CPC A01H 5/10; A01H 6/46

(57) ABSTRACT

Embodiments of the present disclosure pertain to modified plants or seeds that include: (1) overexpressed rice SUMO E3 ligase SIZ1 (OsSIZ1), an analog thereof, a homolog thereof, a derivative thereof, or combinations thereof; and (2) overexpressed Larrea tridentate rubisco activase (LtRCA), an analog thereof, a homolog thereof, a derivative thereof, or combinations thereof. The modified plant or seed demonstrates enhanced resistance to environmental stress. Additional embodiments of the present disclosure pertain to methods of developing a modified plant or seed of the present disclosure by overexpressing in the plant or seed: (1) rice SUMO E3 ligase SIZ1 (OsSIZ1), an analog thereof, a homolog thereof, a derivative thereof, or combinations thereof; and (2) Larrea tridentate rubisco activase (LtRCA), an analog thereof, a homolog thereof, a derivative thereof, or combinations thereof. Further embodiments pertain to methods of growing a modified plant or seed of the present disclosure in a field.

### CROSS-REFERENCE TO RELATED APPLICATIONS

This application claims priority to U.S. Provisional Patent Application No. 63/293,175, filed on Dec. 23, 2021. The entirety of the aforementioned application is incorporated herein by reference.

### STATEMENT UNDER 37 C.F.R. § 1.834(C)(1)

Pursuant to 37 C.F.R. § 1.834, Applicant hereby submits a sequence listing as an XML file (“Sequence Listing”). The name of the file containing the Sequence Listing is “AF13368.P032US.xml”. The date of the creation of the Sequence Listing is Dec. 20, 2022. The size of the Sequence Listing is 8,000 bytes. Applicant hereby incorporates by reference the material in the Sequence Listing.

### BACKGROUND

Crop production is threatened by abiotic stresses worldwide. Heat and drought are major abiotic factors, which account for significant yield losses. The frequency and severity of heat and drought episodes are already on the rise. Therefore, a need exists for developing new strategies to improve multiple stress tolerance in plants.

### SUMMARY

In some embodiments, the present disclosure pertains to a modified plant or seed. In some embodiments, the modified plant or seed includes: (1) overexpressed rice SUMO E3 ligase SIZ1 (OsSIZ1), an analog thereof, a homolog thereof, a derivative thereof, or combinations thereof; and (2) overexpressed Larrea tridentate rubisco activase (LtRCA), an analog thereof, a homolog thereof, a derivative thereof, or combinations thereof. In some embodiments, the modified plant or seed demonstrates enhanced resistance to environmental stress.

In some embodiments, the modified plant or seed includes overexpressed OsSIZ1. In some embodiments, the overexpressed OsSIZ1 includes SEQ ID NO: 1 or a sequence with at least 50% identity with SEQ ID NO: 1. In some embodiments, the modified plant or seed includes an overexpressed analog, homolog or derivative of OsSIZ1. In some embodiments, the analog, homolog or derivative shares at least 50% identity with SEQ ID NO: 1.

In some embodiments, the modified plant or seed includes overexpressed LtRCA. In some embodiments, the overexpressed LtRCA includes SEQ ID NO: 2 or a sequence with at least 50% identity with SEQ ID NO: 2. In some embodiments, the modified plant or seed includes an overexpressed analog, homolog, or derivative of LtRCA. In some embodiments, the analog, homolog or derivative shares at least 50% identity with SEQ ID NO: 2.

In some embodiments, the modified plant or seed includes, without limitation, soybean, maize, sorghum, cotton, alfalfa, rice, wheat, barley, potato, legumes, lettuce, tomato, peas, beans, lentils, peanuts, cucumber, hemp, and brachypodium. In some embodiments, the modified plant or seed includes a modified seed. In some embodiments, the modified plant or seed includes a modified plant.

Additional embodiments of the present disclosure pertain to methods of developing a modified plant or seed of the present disclosure by overexpressing in the plant or seed: (1) rice SUMO E3 ligase SIZ1 (OsSIZ1), an analog thereof, a homolog thereof, a derivative thereof, or combinations thereof; and (2) Larrea tridentate rubisco activase (LtRCA), an analog thereof, a homolog thereof, a derivative thereof, or combinations thereof. In some embodiments, the over-expressing of each of the genes includes modifying the expression of at least one endogenous gene in the plant or seed, introducing at least one exogenous gene into the plant or seed, or combinations thereof.

Additional embodiments of the present disclosure pertain to methods of growing a modified plant or seed of the present disclosure in a field by applying the modified plant or seed to the field. In some embodiments, the applying includes applying the modified seed to the field. In some embodiments, the applying includes applying the modified plant to the field.

### FIGURES

FIGS. 1A-1B provide analyses of wild-type, LtRCA-overexpressing, OsSIZ1-overexpressing, and OsSIZ1/LtRCA co-overexpressing plants under high heat stress conditions. FIG. 1A provides plant height analysis of wild-type and transgenic plants after heat stress treatment. FIG. 1B provides seed yield analysis of wild-type and transgenic plants after high heat stress treatment. WT, wild-type plant; SIZ1, OsSIZ1-overexpressing plant; RCA, LtRCA-overexpressing plant; SR3 to SR5, three independent OsSIZ1/LtRCA co-overexpressing plants. n=15 plants (significant difference at α=0.05 is indicated by different letters).

FIGS. 2A-2B provide analyses of wild-type, LtRCA-overexpressing, OsSIZ1-overexpressing, and OsSIZ1/LtRCA co-overexpressing plants under drought stress conditions. FIG. 2A provides plant height analysis of wild-type and transgenic plants after drought stress treatment. FIG. 2B provides seed yield analysis of wild-type and transgenic plants after drought stress treatment. WT, wild-type plant; SIZ1, OsSIZ1-overexpressing plant; RCA, LtRCA-overexpressing plant; SR2 to SR5, four independent OsSIZ1/LtRCA co-overexpressing plants. n=9 plants (significant difference at α=0.05 is indicated by different letters).

FIG. 3 provides an analysis of wild-type, OsSIZ1-overexpressing, LtRCA-overexpressing, and OsSIZ1/LtRCA co-overexpressing plants on Murashige and Skoog (MS) mediate plates supplemented with polyethylene glycol (PEG). Black bars, MS plate supplemented with PEG on day 1; grey bars, MS plate supplemented with PEG on day 3. WT, wild-type plant; SIZ1, OsSIZ1-overexpressing plant; RCA, LtRCA-overexpressing plant; SR3 to SR5, three independent OsSIZ1/LtRCA co-overexpressing plants. n=10 plants (significant difference at α=0.05 is indicated by different letters).

FIGS. 4A-4B provide analyses of wild-type, OsSIZ1-overexpressing, LtRCA-overexpressing, and OsSIZ1/LtRCA co-overexpressing plants under combined heat and drought stresses. FIG. 4A shows plant height analysis of wild-type and transgenic plants after treatment with combined heat and drought stresses. FIG. 4B shows seed yield analysis of wild-type and transgenic plants after treatment with combined heat and drought stresses. WT, wild-type plant; SIZ1, OsSIZ1-overexpressing plant; RCA, LtRCA-overexpressing plant; SR3 to SR5, three independent OsSIZ1/LtRCA co-overexpressing plants. n=15 plants (significant difference at α=0.05 is indicated by different letters).

FIGS. 5A-5B provide comparisons of the performance of OsSIZ1/RCA co-overexpressing Arabidopsis plants with wild-type and other transgenic Arabidopsis plants under combined heat and drought stresses. FIG. 5A shows plant height analysis of wild-type and transgenic plants under combined heat and drought stresses. FIG. 5B shows seed yield analysis of wild-type and transgenic plants after combined heat and drought treatment. Three-week-old plants under normal growth condition were transferred into a growth chamber that was set at 37° C. for 5.5 h and 22° C. for 18.5 h per day with a photoperiod of 16 h light and 8 h darkness. The irrigation was reduced to half of the amount of water used for control plants under normal growth conditions. WT, wild-type plants; SIZ1, OsSIZ1-overexpressing plants; RCA, RCA-overexpressing plants; SA4, a OsSIZ1/AVP1 co-overexpressing line; SR3 to SR5, three independent OsSIZ1/RCA co-overexpressing lines. n=15 plants (significant difference at α=0.05 is indicated by different letters).

FIG. 6 provides an analysis of wild-type, OsSIZ1-overexpressing, and OsSIZ1/LtRCA co-overexpressing plants under mild salt stress treatment. WT, wild-type plant; SIZ1, OsSIZ1-overexpressing plant; SR1 to SR5, five independent OsSIZ1/LtRCA co-overexpressing plants. n=10 plants (significant difference at α=0.05 is indicated by different letters).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Environmental stresses such as drought and heat cause significant losses in agriculture worldwide. Water availability is the number 1 limiting factor for crop production, but heat wave is now becoming a serious problem. Climate change prediction indicates that many parts of the world are becoming hotter in the summer, including the most productive agricultural land in the US. The combined effects of high temperature and drought have detrimental effects on the growth and productivity of crops, as do the combined effects of heat and salinity.

For instance, cotton production in the United States is facing a challenge that American farmers have never experienced before. To increase cotton's yield, even to sustain its production in the United States, a need exists to develop drought- and heat-tolerant cotton varieties.

One way to increase drought and heat-tolerance in plants (e.g., cotton) is to overexpress genes that confer increased abiotic stress tolerance in the plant, which would lead to substantially higher yield under heat stress and low irrigation conditions. Previously, Applicant demonstrated that OsSIZ1/AVP1 co-overexpressing cotton plants had higher water use efficiency and heat tolerance, which led to a significant increase of cotton fiber for cotton grown in dryland conditions. Plant Biotech. J. 19, 462-476 (2021).

In addition, Applicant previously created another transgenic cotton line, RCA/AVP1 co-overexpressing cotton. Applicant showed that this line was also very tolerant to drought and heat stresses, and it outperformed wild-type cotton under combined drought and heat stresses, as well as in field conditions.

Nonetheless, in view of increasing frequencies and severities of heat and drought episodes, a need remains for developing new strategies to improve multiple stress tolerance in plants. For instance, drought and heat are major environmental stresses that limit cotton production in many regions, such as the Texas High Plains. Numerous embodiments of the present disclosure aim to address the aforementioned need.

### Modified Plants and Seeds

In some embodiments, the present disclosure pertains to modified plants, modified seeds, or combinations thereof. In some embodiments, the modified plants and seeds of the present disclosure include: (1) overexpressed rice SUMO E3 ligase SIZ1 (OsSIZ1), an analog thereof, a homolog thereof, a derivative thereof, or combinations thereof; and (2) overexpressed Larrea tridentate rubisco activase (LtRCA), an analog thereof, a homolog thereof, a derivative thereof, or combinations thereof. In some embodiments, the modified plant or seed demonstrates enhanced resistance to environmental stresses. As set forth in more detail herein, the modified plants and seeds of the present disclosure may include various overexpressed genes. Moreover, the modified plants and seeds of the present disclosure may demonstrate enhanced resistance to various sources of environmental stress.

### Overexpressed Genes

The modified plants and seeds of the present disclosure may include various types of overexpressed genes. For instance, in some embodiments, the modified plant or seed includes overexpressed OsSIZ1. In some embodiments, the overexpressed OsSIZ1 includes SEQ ID NO: 1.

In some embodiments, the overexpressed OsSIZ1 includes a sequence with at least 50% identity with SEQ ID NO: 1. In some embodiments, the overexpressed OsSIZ1 includes a sequence with at least 55% identity with SEQ ID NO: 1. In some embodiments, the overexpressed OsSIZ1 includes a sequence with at least 60% identity with SEQ ID NO: 1. In some embodiments, the overexpressed OsSIZ1 includes a sequence with at least 65% identity with SEQ ID NO: 1. In some embodiments, the overexpressed OsSIZ1 includes a sequence with at least 70% identity with SEQ ID NO: 1. In some embodiments, the overexpressed OsSIZ1 includes a sequence with at least 75% identity with SEQ ID NO: 1. In some embodiments, the overexpressed OsSIZ1 includes a sequence with at least 80% identity with SEQ ID NO: 1. In some embodiments, the overexpressed OsSIZ1 includes a sequence with at least 85% identity with SEQ ID NO: 1. In some embodiments, the overexpressed OsSIZ1 includes a sequence with at least 90% identity with SEQ ID NO: 1. In some embodiments, the overexpressed OsSIZ1 includes a sequence with at least 95% identity with SEQ ID NO: 1.

In some embodiments, the modified plant or seed includes an overexpressed analog of OsSIZ1. In some embodiments, the analog shares at least 50% identity with SEQ ID NO: 1. In some embodiments, the analog shares at least 55% identity with SEQ ID NO: 1. In some embodiments, the analog shares at least 60% identity with SEQ ID NO: 1. In some embodiments, the analog shares at least 65% identity with SEQ ID NO: 1. In some embodiments, the analog shares at least 70% identity with SEQ ID NO: 1. In some embodiments, the analog shares at least 75% identity with SEQ ID NO: 1. In some embodiments, the analog shares at least 80% identity with SEQ ID NO: 1. In some embodiments, the analog shares at least 85% identity with SEQ ID NO: 1. In some embodiments, the analog shares at least 90% identity with SEQ ID NO: 1. In some embodiments, the analog shares at least 95% identity with SEQ ID NO: 1.

In some embodiments, the modified plant or seed includes an overexpressed homolog of OsSIZ1. In some embodiments, the homolog shares at least 50% identity with SEQ ID NO: 1. In some embodiments, the homolog shares at least 55% identity with SEQ ID NO: 1. In some embodiments, the homolog shares at least 60% identity with SEQ ID NO: 1. In some embodiments, the homolog shares at least 65% identity with SEQ ID NO: 1. In some embodiments, the homolog shares at least 70% identity with SEQ ID NO: 1. In some embodiments, the homolog shares at least 75% identity with SEQ ID NO: 1. In some embodiments, the homolog shares at least 80% identity with SEQ ID NO: 1. In some embodiments, the homolog shares at least 85% identity with SEQ ID NO: 1. In some embodiments, the homolog shares at least 90% identity with SEQ ID NO: 1. In some embodiments, the homolog shares at least 95% identity with SEQ ID NO: 1.

In some embodiments, the modified plant or seed includes an overexpressed derivative of OsSIZ1. In some embodiments, the derivative shares at least 50% identity with SEQ ID NO: 1. In some embodiments, the derivative shares at least 55% identity with SEQ ID NO: 1. In some embodiments, the derivative shares at least 60% identity with SEQ ID NO: 1. In some embodiments, the derivative shares at least 65% identity with SEQ ID NO: 1. In some embodiments, the derivative shares at least 70% identity with SEQ ID NO: 1. In some embodiments, the derivative shares at least 75% identity with SEQ ID NO: 1. In some embodiments, the derivative shares at least 80% identity with SEQ ID NO: 1. In some embodiments, the derivative shares at least 85% identity with SEQ ID NO: 1. In some embodiments, the derivative shares at least 90% identity with SEQ ID NO: 1. In some embodiments, the derivative shares at least 95% identity with SEQ ID NO: 1.

In some embodiments, the modified plant or seed includes overexpressed LtRCA. In some embodiments, the overexpressed LtRCA includes SEQ ID NO: 2.

In some embodiments, the overexpressed LtRCA includes a sequence with at least 50% identity with SEQ ID NO: 2. In some embodiments, the overexpressed LtRCA includes a sequence with at least 55% identity with SEQ ID NO: 2. In some embodiments, the overexpressed LtRCA includes a sequence with at least 60% identity with SEQ ID NO: 2. In some embodiments, the overexpressed LtRCA includes a sequence with at least 65% identity with SEQ ID NO: 2. In some embodiments, the overexpressed LtRCA includes a sequence with at least 70% identity with SEQ ID NO: 2. In some embodiments, the overexpressed LtRCA includes a sequence with at least 75% identity with SEQ ID NO: 2. In some embodiments, the overexpressed LtRCA includes a sequence with at least 80% identity with SEQ ID NO: 2. In some embodiments, the overexpressed LtRCA includes a sequence with at least 85% identity with SEQ ID NO: 2. In some embodiments, the overexpressed LtRCA includes a sequence with at least 90% identity with SEQ ID NO: 2. In some embodiments, the overexpressed LtRCA includes a sequence with at least 95% identity with SEQ ID NO: 2.

In some embodiments, the modified plant or seed includes an overexpressed analog of LtRCA. In some embodiments, the analog shares at least 50% identity with SEQ ID NO: 2. In some embodiments, the analog shares at least 55% identity with SEQ ID NO: 2. In some embodiments, the analog shares at least 60% identity with SEQ ID NO: 2. In some embodiments, the analog shares at least 65% identity with SEQ ID NO: 2. In some embodiments, the analog shares at least 70% identity with SEQ ID NO: 2. In some embodiments, the analog shares at least 75% identity with SEQ ID NO: 2. In some embodiments, the analog shares at least 80% identity with SEQ ID NO: 2. In some embodiments, the analog shares at least 85% identity with SEQ ID NO: 2. In some embodiments, the analog shares at least 90% identity with SEQ ID NO: 2. In some embodiments, the analog shares at least 95% identity with SEQ ID NO: 2.

In some embodiments, the modified plant or seed includes an overexpressed homolog of LtRCA. In some embodiments, the homolog shares at least 50% identity with SEQ ID NO: 2. In some embodiments, the homolog shares at least 55% identity with SEQ ID NO: 2. In some embodiments, the homolog shares at least 60% identity with SEQ ID NO: 2. In some embodiments, the homolog shares at least 65% identity with SEQ ID NO: 2. In some embodiments, the homolog shares at least 70% identity with SEQ ID NO: 2. In some embodiments, the homolog shares at least 75% identity with SEQ ID NO: 2. In some embodiments, the homolog shares at least 80% identity with SEQ ID NO: 2. In some embodiments, the homolog shares at least 85% identity with SEQ ID NO: 2. In some embodiments, the homolog shares at least 90% identity with SEQ ID NO: 2. In some embodiments, the homolog shares at least 95% identity with SEQ ID NO: 2.

In some embodiments, the modified plant or seed includes an overexpressed derivative of LtRCA. In some embodiments, the derivative shares at least 50% identity with SEQ ID NO: 2. In some embodiments, the derivative shares at least 55% identity with SEQ ID NO: 2. In some embodiments, the derivative shares at least 60% identity with SEQ ID NO: 2. In some embodiments, the derivative shares at least 65% identity with SEQ ID NO: 2. In some embodiments, the derivative shares at least 70% identity with SEQ ID NO: 2. In some embodiments, the derivative shares at least 75% identity with SEQ ID NO: 2. In some embodiments, the derivative shares at least 80% identity with SEQ ID NO: 2. In some embodiments, the derivative shares at least 85% identity with SEQ ID NO: 2. In some embodiments, the derivative shares at least 90% identity with SEQ ID NO: 2. In some embodiments, the derivative shares at least 95% identity with SEQ ID NO: 2.

### Forms of Overexpressed Genes

Overexpressed genes in the modified plants and seeds of the present disclosure may be in various forms. For instance, in some embodiments, each of the overexpressed genes includes, without limitation, an endogenous gene, a transgene, and combinations thereof.

In some embodiments, at least one of the overexpressed genes includes a transgene. In some embodiments, each of the overexpressed genes includes a transgene. In some embodiments, the transgene is positioned on an expression vector.

In some embodiments, the overexpressed genes include transgenes that are positioned on one or more expression vectors. In some embodiments, the transgenes are positioned on the same expression vector.

In some embodiments, at least one of the overexpressed genes includes an endogenous gene. In some embodiments, a promoter of the endogenous gene has been modified to overexpress the endogenous gene.

### Modified Plants and Seeds

The modified plants and seeds of the present disclosure can include various species. For instance, in some embodiments, the modified plants and seeds of the present disclosure include soybean, maize, sorghum, cotton, alfalfa, rice, wheat, barley, potato, legumes (e.g., alfalfa and medicago), lettuce, tomato, peas, beans, lentils, peanuts, cucumber, hemp, and brachypodium.

In some embodiments, the modified plants and seeds of the present disclosure include corn. In some embodiments, the modified plants and seeds of the present disclosure include cotton. In some embodiments, the modified plants and seeds of the present disclosure include soybean. In some embodiments, the modified plants and seeds of the present disclosure include medicago.

In some embodiments, the modified plants and seeds of the present disclosure include a modified seed. In some embodiments, the modified plants and seeds of the present disclosure include a modified plant.

### Enhanced Resistance to Environmental Stress

The modified plants and seeds of the present disclosure have enhanced resistance to various types of environmental stress. For instance, in some embodiments, the environmental stress includes, without limitation, heat stress, drought stress, high salt concentrations, low temperatures (e.g., temperatures at or below freezing), and combinations thereof.

In some embodiments, the environmental stress includes heat stress and drought stress. In some embodiments, the environmental stress includes heat stress, drought stress, low temperature stress, and high salt concentrations.

In some embodiments, the environmental stress includes heat stress. In some embodiments, heat stress is defined as temperatures of at least about 10° C. above an optimum temperature for the plant or seed for at least two consecutive days. In some embodiments, heat stress is defined as temperatures of at least about 10° C. above an optimum temperature for the plant or seed for at least one week. In some embodiments, heat stress is defined as temperatures of at least about 10° C. above an optimum temperature for the plant or seed for at least two weeks.

In some embodiments, the environmental stress includes low temperatures. In some embodiments, low temperature is defined as temperatures of at least about 10° C. below an optimum temperature for the plant or seed for at least two consecutive days. In some embodiments, low temperature is defined as temperatures of at least about 10° C. below an optimum temperature for the plant or seed for at least one week. In some embodiments, low temperature is defined as temperatures of at least about 10° C. below an optimum temperature for the plant or seed for at least two weeks. In some embodiments, low temperature includes temperatures below freezing.

In some embodiments, the environmental stress includes drought stress. In some embodiments, the drought stress is defined as lack of water for at least one week. In some embodiments, the drought stress is defined as lack of water for at least ten days. In some embodiments, the drought stress is defined as lack of water for at least two weeks.

In some embodiments, the environmental stress includes a high salt concentration (e.g., high concentrations of sodium chloride). In some embodiments, the high salt concentration is defined as a soil salt concentration of at least about 50 mM. In some embodiments, the high salt concentration is defined as a soil salt concentration of at least about 75 mM. In some embodiments, the high salt concentration is defined as a soil salt concentration of at least about 100 mM.

The enhanced resistance to environmental stress in the plants and seeds of the present disclosure can have various effects on the plants and seeds. For instance, in some embodiments, the enhanced resistance to environmental stress is defined by at least one of enhanced crop yield relative to unmodified plants or seeds, enhanced seed yield relative to unmodified plants, display of longer hypocotyl lengths relative to unmodified plants, enhanced growth rates relative to unmodified plants or seeds, and combinations thereof.

In some embodiments, the enhanced resistance to environmental stress is defined by at least enhanced crop yield relative to unmodified plants or seeds. In some embodiments, the enhanced crop yield includes an enhancement of at least 10% relative to unmodified plants or seeds. In some embodiments, the enhanced crop yield includes an enhancement of at least 20% relative to unmodified plants or seeds. In some embodiments, the enhanced crop yield includes an enhancement of at least 30% relative to unmodified plants or seeds. In some embodiments, the enhanced crop yield includes an enhancement of at least 40% relative to unmodified plants or seeds. In some embodiments, the enhanced crop yield includes an enhancement of at least 50% relative to unmodified plants or seeds. In some embodiments, the enhanced crop yield includes an enhancement of at least 100% relative to unmodified plants or seeds. In some embodiments, the enhanced crop yield includes an enhancement of at least 150% relative to unmodified plants or seeds. In some embodiments, the enhanced crop yield includes an enhancement of at least 200% relative to unmodified plants or seeds.

In some embodiments, the enhanced resistance to environmental stress is defined by at least enhanced growth rates relative to unmodified plants or seeds. In some embodiments, the enhanced growth rates include an enhancement of at least 10% relative to unmodified plants or seeds. In some embodiments, the enhanced growth rates include an enhancement of at least 20% relative to unmodified plants or seeds. In some embodiments, the enhanced growth rates include an enhancement of at least 30% relative to unmodified plants or seeds. In some embodiments, the enhanced growth rates include an enhancement of at least 40% relative to unmodified plants or seeds. In some embodiments, the enhanced growth rates include an enhancement of at least 50% relative to unmodified plants or seeds. In some embodiments, the enhanced growth rates include an enhancement of at least 100% relative to unmodified plants or seeds. In some embodiments, the enhanced growth rates include an enhancement of at least 150% relative to unmodified plants or seeds. In some embodiments, the enhanced growth rates include an enhancement of at least 200% relative to unmodified plants or seeds.

In some embodiments, the enhanced resistance to environmental stress is defined by at least enhanced seed yield relative to unmodified plants. In some embodiments, the enhanced seed yield includes an enhancement of at least 10% relative to unmodified plants. In some embodiments, the enhanced seed yield includes an enhancement of at least 20% relative to unmodified plants. In some embodiments, the enhanced seed yield includes an enhancement of at least 30% relative to unmodified plants. In some embodiments, the enhanced seed yield includes an enhancement of at least 40% relative to unmodified plants. In some embodiments, the enhanced seed yield includes an enhancement of at least 50% relative to unmodified plants. In some embodiments, the enhanced seed yield includes an enhancement of at least 100% relative to unmodified plants. In some embodiments, the enhanced seed yield includes an enhancement of at least 150% relative to unmodified plants. In some embodiments, the enhanced seed yield includes an enhancement of at least 200% relative to unmodified plants.

### Methods of Developing Modified Plants and Seeds

Additional embodiments of the present disclosure pertain to methods of developing the modified plants and seeds of the present disclosure. In some embodiments, such methods include: (1) overexpressing OsSIZ1, an analog thereof, a homolog thereof, a derivative thereof, or combinations thereof in a plant or seed; and (2) overexpressing LtRCA, an analog thereof, a homolog thereof, a derivative thereof, or combinations thereof, in the plant or seed to form the modified plant or seed. As such, the modified plant or seed demonstrates enhanced resistance to environmental stress.

Various methods may be utilized to over-express genes. For instance, in some embodiments, the over-expressing of each of the genes includes modifying the expression of at least one endogenous gene in the plant or seed, introducing at least one exogenous gene into the plant or seed, or combinations thereof.

In some embodiments, the over-expressing of one or more of the genes includes introducing at least one exogenous gene into the plant or seed. In some embodiments, the introducing occurs by a method that includes, without limitation, transferred DNA insertion, enhancer trap insertion, floral-dip transformation, callus transformation, tissue transformation, mobile genetic elements insertion, activation tagging insertion, fox hunting insertion, particle bombardment, and combinations thereof. In some embodiments, the introducing occurs by floral-dip transformation. In some embodiments, the introducing occurs at a seedling stage or an adult stage of a plant.

### Methods of Growing Modified Plants and Seeds

Additional embodiments of the present disclosure pertain to methods of growing modified plants and seeds of the present disclosure in a field. In some embodiments, such methods include applying the modified plant or seed to the field. In some embodiments, the applying includes applying the modified seed to the field. In some embodiments, the applying includes applying the modified plant to the field.

The modified plants and seeds of the present disclosure may be grown in various fields. For instance, in some embodiments, the field includes a field vulnerable to environmental stress. In some embodiments, the environmental stress includes, without limitation, heat stress, drought stress, high salt concentrations, low temperatures, and combinations thereof.

In some embodiments, the modified plant or seed demonstrates enhanced resistance to environmental stress. In some embodiments, the enhanced resistance to environmental stress is defined by at least one of enhanced crop yield relative to unmodified plants or seeds, enhanced seed yield relative to unmodified plants, display of longer hypocotyl lengths relative to unmodified plants, enhanced photosynthetic rates relative to unmodified plants or seeds, enhanced growth rates relative to unmodified plants or seeds, and combinations thereof.

### Applications and Advantages

The modified plants and seeds of the present disclosure can have numerous advantages. In particular, co-overexpression of RCA and OsSIZ1 improves plant and seed performance under environmental stress conditions and increase seed yield several folds under combined drought and heat conditions.

As such, the modified plants and seeds of the present disclosure can have numerous applications. For instance, in some embodiments, the modified plants and seeds of the present disclosure could double or triple crop yield for dryland agricultural regions of the world, which is about half of the world's arable land. In particular, the growth of the modified plants and seeds of the present disclosure in semiarid and arid regions of the world could provide food security to about 50% of the world's population in developing countries and substantially increase a farmer's income in numerous countries, such as the United States, Australia, and Brazil. The modified plants and seeds of the present disclosure could also allow farmers to use much less water for irrigation and less fertilizers for crop production, which could in turn help make food production sustainable worldwide.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### EXAMPLE 1. CO-OVEREXPRESSION OF OsSSIZ1 AND LtRCA IN ARABIDOPSIS THALIANA TO FURTHER IMPROVE HEAT AND DROUGHT TOLERANCE

The rice gene OsSIZ1 has been shown to confer increased tolerance to heat, drought, and salt stresses when overexpressed in transgenic plants. The RCA gene from Larrea tridentata, LtRCA has also been shown to improve heat tolerance in Arabidopsis. In this Example, Applicant co-overexpressed OsSIZ1 and LtRCA in Arabidopsis thaliana with the intention of further improving heat and drought tolerance. The results indicated that co-overexpression of OsSIZ1 and LtRCA significantly improved plant tolerance to heat stress and combined heat and drought stresses. Most importantly, OsSIZ1 and LtRCA co-overexpressing plants were able to generate seed yield that was several magnitudes higher than that of wild-type plants under heat and drought stresses. The findings of this study would be useful to improve heat and drought tolerance in commercially important crops in the future.

### Example 1.1. OsSIZ1/LtRCA Co-Overexpression Further Increases Heat Tolerance in Arabidopsis Plants

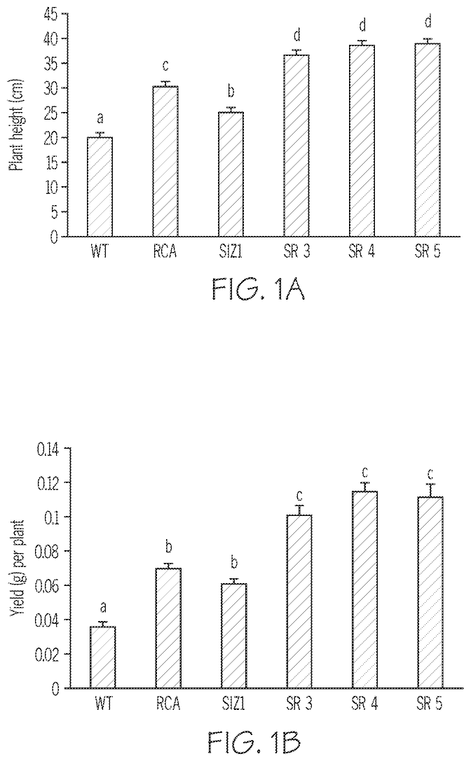

To test if OsSIZ1/LtRCA co-overexpressing plants were more heat tolerant than wild-type plants, heat stress experiments were conducted with plants grown on MS plates and plants grown in soil under moderate and high heat stress conditions. Under normal growth conditions, wild-type plants and transgenic plants grown in soil did not show much difference morphologically. For high heat stress treatment, three-week-old wild-type and transgenic plants were transferred to a growth chamber that was set at 37° C. for 5.5 hours per day and 22° C. for the rest of the day. After heat treatment for 45 days, all OsSIZ1/LtRCA co-overexpressing plants exhibited significantly taller height than all other genotypes being tested (FIG. 1A). A greater number of siliques was also observed compared to wild-type and single gene overexpressing plants. OsSIZ1-overexpressing plants and LtRCA-overexpressing plants also performed better than wild-type plants (FIG. 1A). More importantly, OsSIZ1/LtRCA co-overexpressing plants generated almost 150% higher seed yield than wild-type plants, and nearly a 2-fold increase compared to OsSIZ1-overexpressing and LtRCA-overexpressing plants (FIG. 1B). This indicates that the OsSIZ1 and LtRCA genes have acted synergistically under heat stress conditions to show a superior performance in OsSIZ1/LtRCA co-overexpressing plants. Even though LtRCA-overexpressing plants exhibited significantly taller heights than OsSIZ1-overexpressing plants, no significant difference was observed between their seed yields.

### Example 1.2. OsSIZ1/LtRCA Co-Overexpressing Plants are More Drought Tolerant

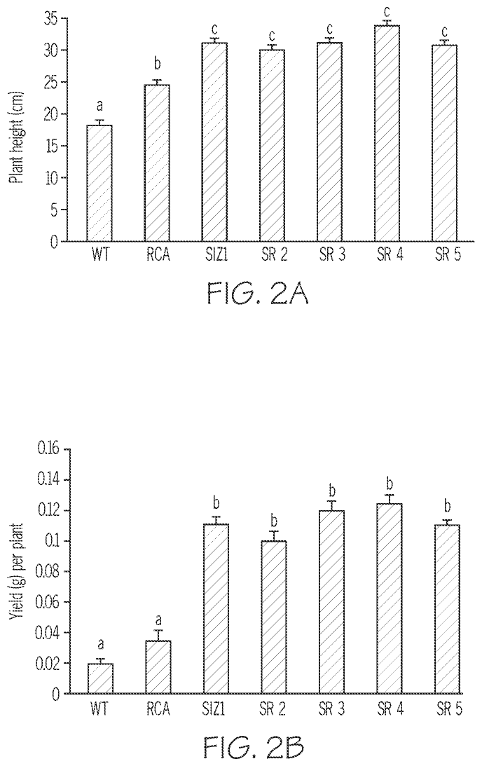

To test if OsSIZ1/LtRCA co-overexpressing plants were more drought tolerant than wild-type plants, drought stress experiments were conducted with soil grown plants. Wild-type, OsSIZ1-overexpressing, LtRCA-overexpressing, and OsSIZ1/LtRCA co-overexpressing plants (4 independent lines SR2, SR3, SR4, and SR5) were fully irrigated until three weeks old. Then irrigation was completely withheld for two weeks. Under drought stress treatment OsSIZ1/LtRCA co-overexpressing and OsSIZ1 overexpressing plants displayed better phenotypes than wild-type and LtRCA-overexpressing plants. Although LtRCA-overexpressing plants grew taller than wild-type plants (FIG. 2A), their yield performance was not significantly better than wild-type plants (FIG. 2B).

### Example 1.3. OsSIZ1/LtRCA Co-Overexpressing Plants are More Tolerant to Water Deficit Stress

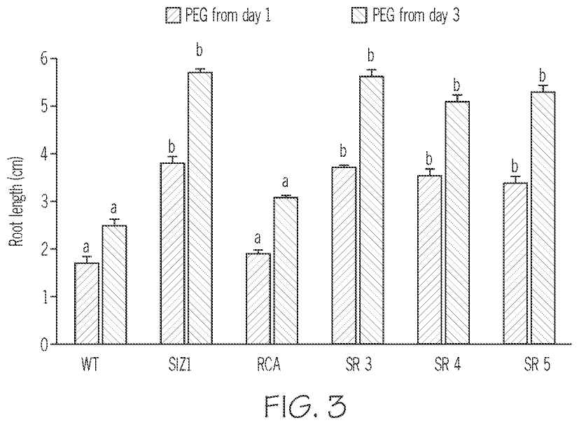

To test if OsSIZ1/LtRCA co-overexpressing plants were more tolerant to water deficit stress than wild-type plants, water deficit conditions were created by supplementing Murashige and Skoog (MS) media plates with polyethylene glycol-8000 (PEG-8000). PEG, being a high molecular weight compound, is known to affect osmotic pressure and reduce water potential in the growth media, thereby inducing a dehydration stress condition for plants grown on MS media. PEG was therefore used to test the effect of water deficit on early plant growth. Seeds were either directly plated on MS media containing PEG, or three-day-old seedlings were transferred to MS plates supplemented with PEG. For both type of treatments OsSIZ1/LtRCA co-overexpressing plants and OsSIZ1-overexpressing plants displayed the longest root lengths whereas wild-type and LtRCA-overexpressing plants displayed limited root growth that was nearly 50% shorter than OsSIZ1/LtRCA co-overexpressing plants and OsSIZ1-overexpressing plants (FIG. 3).

### Example 1.4. OsSIZ1/LtRCA Co-Overexpressing Plants are More Tolerant to Combined Heat and Drought Stresses

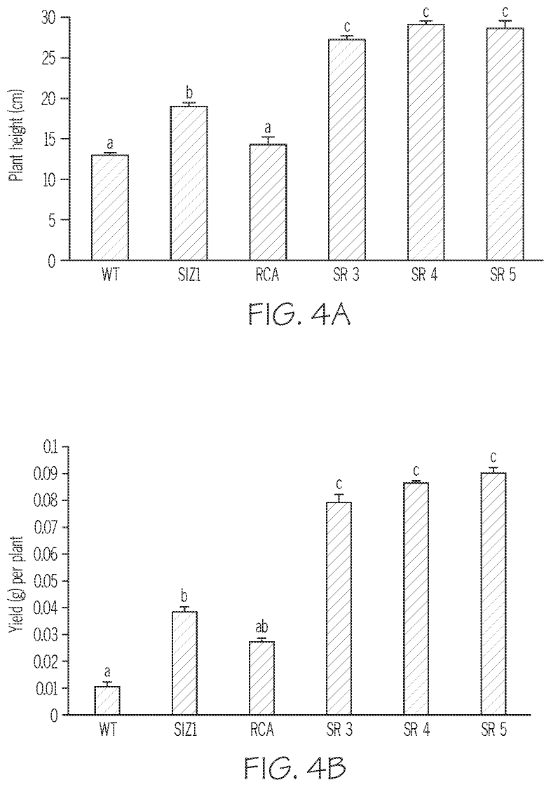

Temperature and water deficit stress usually occur together. Therefore, it was of great importance to assess the performance of OsSIZ1/LtRCA co-overexpressing plants when they were subjected to combined stresses of heat and drought. To test how OsSIZ1/LtRCA co-overexpressing plants would perform under combined heat and drought stresses, three-week-old plants grown under normal growth condition were transferred to a heat chamber and irrigation was reduced to half. Applicant's results show that OsSIZ1/LtRCA co-overexpressing plants outperformed all other plants by big margins (FIG. 4A).

The OsSIZ1-overexpressing plants performed significantly better than wild-type and LtRCA-overexpressing plants. However, such performance was not comparable to OsSIZ1/LtRCA co-overexpressing plants, which produced a seed yield that was 10-fold higher than wild-type plants, and more than a 100% yield increase compared to OsSIZ1-overexpressing and LtRCA-overexpressing plants (FIG. 4B).

Comparing this with the results of single stress experiments (i.e., heat stress or drought stress alone), it is clear that the biggest difference between the performances of wild-type plants and OsSIZ1/LtRCA co-overexpressing plants were observed under the combined drought and heat stresses. Therefore, the two genes function synergistically in conferring the increased tolerance under combined heat and drought stresses.

### Example 1.5. OsSIZ1/LtRCA Co-Overexpressing Plants Outperform OsSIZ1/AVP1 Co-Overexpressing Plants

Applicant previously showed that overexpression of a gene called AVP1 could increase drought- and salt-tolerance in transgenic cotton. Applicant also showed that overexpression of another gene called OsSIZ1 in cotton dramatically improved drought- and heat-tolerance and significantly increased fiber yields under reduced irrigation and dryland conditions. Applicant then hypothesized that co-overexpression of OsSIZ1 and AVP1 might have a synergistic effect, which could result in higher tolerance to drought, heat, and salt stresses than either AVP1-overexpression or OsSIZ1-overexpression alone, thereby leading to further increased fiber yields under dryland conditions. Indeed, Applicant's experiments with OsSIZ1/AVP1 co-overexpressing cotton confirmed the prediction that Applicant could drastically increase fiber yield under combined drought and heat stresses in greenhouse and even double fiber yield for cotton grown under dryland conditions, such as conditions in Texas High Plains.

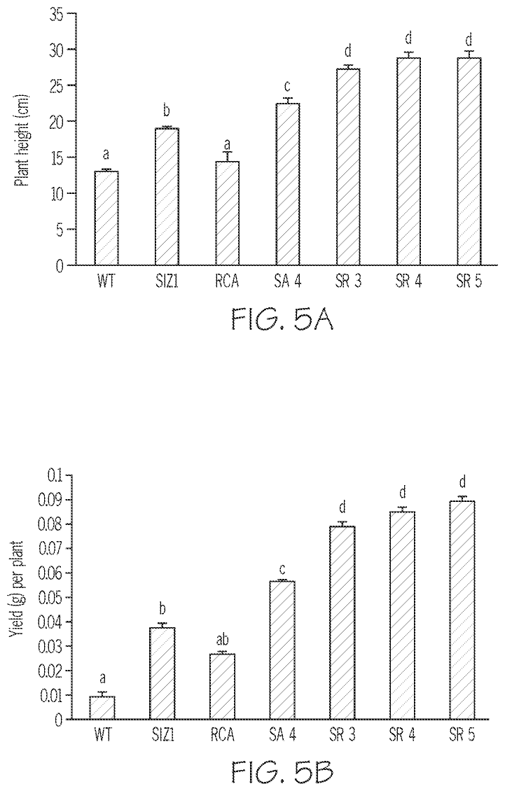

However, as illustrated in FIGS. 5A-5B, Applicant unexpectedly discovered that the OsSIZ1+LtRCA combination works even better than the OsSIZ1+AVP1 combination in conferring increased abiotic stress tolerance. In particular, OsSIZ1/LtRCA co-overexpressing Arabidopsis plants produced at least 50% more seeds than OsSIZ1/AVP1 co-overexpressing Arabidopsis plants under combined drought and heat stresses.

### Example 1.6. OsSIZ1/LtRCA Co-Overexpressing Plants are More Salt Tolerant than Wild-Type Plants

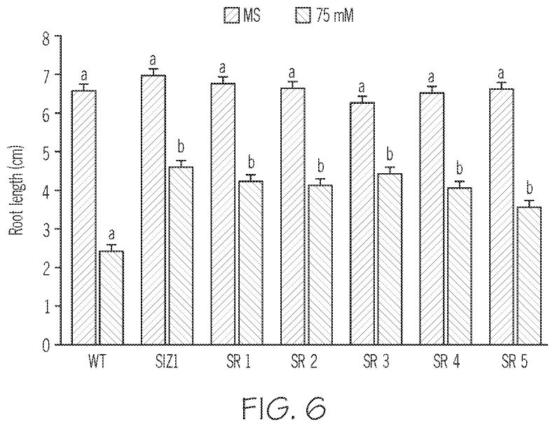

To test the salt tolerance capacity of OsSIZ1/LtRCA co-overexpressing plants, wild-type and transgenic Arabidopsis seeds were sown on MS plates supplemented with NaCl at either 75 mM or 100 mM. The OsSIZ1/LtRCA co-overexpressing plants performed significantly better than wild-type plants (FIG. 6). However, no difference was observed between OsSIZ1-overexpressing plants and OsSIZ1/LtRCA co-overexpressing plants.

### Example 1.7. Vector Construction

To develop an expression cassette containing genes of interest, the binary vector pBI121 was used as the backbone vector. The neomycin phosphotransferase II gene (NPTII) driven by the nopaline synthase promoter, which confers kanamycin resistance in transgenic cells, was chosen as the selection marker for transgenic plant identification.

Initially, the 35S-GUS expression cassette in pBI121 was replaced by a short 70 bp linker sequence from the vector pJG4-5 using the restriction enzymes Eco RI and Hind III to generate the vector pBI121e. The pHL080, which contains the SUMO E3 ligase gene OsSIZ1 driven by the maize ubiquitin promoter, was utilized. The pUbi-OsSIZ1 expression cassette from pHL080 was then isolated using Hind III partial digestion and inserted into the Hind III site of pBI121e. The resultant vectors with the ubiquitin promoter close to the kanamycin resistance gene were selected as pBI121-S+.

To construct the LtRCA expression cassette driven by the light inducible CAB3 promoter, a pBI121 vector with the CAB3 promoter in place of the 35S promoter was digested by Bam HI and Sac I and linked to a small fragment to generate pBI121c without the GUS coding sequence. Then the LtRCA coding sequence was isolated from pUC-RCA using Barn HI and inserted into the Barn HI site of pBI121c to generate pBI121-R. The pCAB3-RCA expression cassette was isolated from pBI121-R using Eco RI and inserted into the Eco RI site of pBI121-S+ to generate pBI121-SR, the OsSIZ1/LtRCA co-overexpressing vector.

### Example 1.8. Agrobacterium-Mediated Plant Transformation

Agrobacterium strain GV3101 was used for Arabidopsis transformation. The binary vector containing the pUbi::OsSIZ1/pCab3::LtRCA construct was introduced into the Agrobacterial strain GV3101 by using a freeze thaw method. Thereafter, the Agrobacterium strain GV3101 carrying the transgene vector was used to transform wild-type (WT) Arabidopsis plants (ecotype Columbia), using the floral-dip technique. Transformed Agrobacteria were cultured in sterilized liquid Luria-Bertani (LB) medium supplemented with 50 μg/ml kanamycin, 50 μg/ml rifampicin, and 20 μg/ml gentamycin. The culture was then incubated overnight in a shaker set at 250 rpm at 28° C. until its optical density (OD) reached the approximate level of OD600=0.8. The Agrobacterial cells were harvested by centrifugation at 3000 g for 15 min at room temperature.

The harvested cells were resuspended into 300 ml of inoculation medium containing 5% sucrose and 0.025% Silwet L-77 surfactant. Meanwhile, WT Arabidopsis plants were grown up to the flowering stage (usually until four weeks old) in a growth chamber set at 22° C. When preparing soil pots, it was made sure that soil was added up to the rim of the pots in order to avoid the soil from falling into the inoculation medium during subsequent floral dipping. Bolting WT plants were transformed by inverting the plants and submerging the above-ground parts of plants in the transformation suspension for about 3 min. For higher efficacy, transformation was carried out in a vacuum jar (13 in Hg). The treated plants were laid horizontally and covered with a plastic film to provide high humidity conditions and were kept in darkness or low light overnight. The plastic films were removed 24 h later. Loose plants in a pot were tied up using skews and tape to keep plants of each pot together and separated from nearby pots. The entire plant transformation process was repeated one week later. Plants were grown under normal growth conditions until reaching the harvesting stage.

### Example 1.9. Transgenic Plant Identification

The seeds harvested from the treated plants (T0 generation) were surface sterilized and plated on Murashige and Skoog (MS) media containing 30 μg/ml kanamycin. The T1 plants exhibiting kanamycin resistance were selected and transferred into soil and grown to obtain T2 seeds. Then the T2 seeds were subjected to screening on MS media plates supplemented with kanamycin to identify putative single insertion lines that exhibit a 3:1 ratio for kanamycin resistance to kanamycin sensitivity. The selected T2 plants were transferred to soil to obtain T3 seeds. The homozygous transgenic plants identified from the kanamycin screening were used for further analysis and verification.

### Example 1.10. Physiological Experiments

Physiological experiments were conducted under two different conditions: seedlings grown on MS media plates and plants grown in soil. The following growth conditions were maintained prior to all the treatments. Arabidopsis seeds were surface sterilized and stored at 4° C. for stratification for four days, following which seeds were plated on the MS media. Then, the seedlings grown on plates were subject to stress treatments accordingly. For soil experiments, plants were grown in soil under normal growth conditions in growth chambers (ENCONAIR AC-60, Ecological chamber Inc., Canada) with 16 h/8 h light/dark photoperiod (light intensity at 120 mmol s−1 m−2), at 22° C., 50% relative humidity and with regular irrigation. Then, three-week-old WT, OsSIZ1-overexpressing, LtRCA-overexpressing, and OsSIZ/LtRCA co-overexpressing plants were subject to individual as well as combined stress treatments, while the control plants were allowed to grow under normal growth conditions. For all the soil experiments, seed collectors were used to minimize possible seed loss prior to harvesting.

### Example 1.11. Heat Stress Treatment

Sterilized seeds of WT, OsSIZ1-overexpressing, LtRCA-overexpressing, and OsSIZ1/LtRCA co-overexpressing plants were sown on MS plates and kept at 30° C. and 37° C. (5.5 hours per day) for moderate and high heat stresses, respectively. These plants were grown vertically for about ten days or until a phenotypic difference was visible. Meanwhile an ambient temperature of 22° C. was maintained for the plates kept under normal growth conditions. Following the treatments, root lengths were measured for all seedlings. Heat tolerance was also tested with soil grown plants. Three-week-old plants of WT, OsSIZ1-overexpressing, LtRCA-overexpressing, and OsSIZ1/LtRCA co-overexpressing plants were transferred into a growth chamber that was set at 37° C. for 5.5 h per day and 22° C. for the rest of the day (16 h/8 h, light/dark photoperiod). Plants were irrigated regularly until the end of the experiment. Then, the plant height and seed yield were recorded. The phenotypes were also documented throughout the experiment. For the soil experiments fifteen biological replicates were used per genotype and the experiments were performed three times. To assess the impact of heat treatment on seed quality, germination rates of the seeds harvested from heat treated plants were also analyzed. An additional heat treatment was conducted while maintaining the same micro-environment for all the genotypes.

### Example 1.12. Drought Stress Treatment

To test how OsSIZ1/LtRCA co-overexpressing plants would perform under drought stress conditions, the following experiment was carried out. Plants were grown in soil under normal growth conditions and were fully irrigated until three weeks old. Then irrigation was completely stopped for two weeks, following which the phenotypes were documented. After that, plants were irrigated again and then allowed to recover. At the end of the experiment, seed yield and plant height were measured. The phenotypes were also documented. Nine biological replicates were used per genotype. The experiment was repeated twice.

### Example 1.13. Combined Heat and Drought Stress Treatment

To test how OsSIZ1/LtRCA co-overexpressing plants would perform under combined heat and drought stresses, the following experiment was carried out. Three-week-old soil grown plants under normal growth conditions were transferred into a growth chamber that was set at 37° C. for 5.5 h per day and 22° C. for the rest of the day (16 h/8 h, light/dark photoperiod). Then, irrigation was reduced to half of the amount of water used for plants under normal growth conditions. For example, those plants were watered once every 3-4 days. The treatment was continued for a maximum of four weeks following which the plants were transferred to normal growth conditions for recovery. Then the seed yield and plant height were measured, and the phenotypes were documented. Nine replicates were used per genotype and the experiment was repeated twice.

### Example 1.14. Water Deficit Treatment with Polyethylene Glycol

To create water deficit stress conditions for seedlings grown on MS plates, polyethylene glycol-8000 (PEG) was used to create water deficit/dehydration stress. PEG is known to reduce water potential in the growth media, thereby inducing a dehydration stress in plant root cells. Therefore, PEG-8000 was used to test how OsSIZ1/LtRCA co-overexpressing plants would perform under water deficit conditions at the seedling stage. Seeds were plated on MS media containing 40% PEG to examine the effect of water deficit stress on seed germination. Germination data and root length were recorded after two weeks. Germination rates and the root lengths of transgenic plants were compared with plants under normal growth condition where the Arabidopsis seeds were plated on MS media.

### Example 1.15. Salt Stress Treatment

For salt stress treatment on plates, three-day old seedlings on MS plates were transferred to MS plates supplemented with 75 or 100 mM NaCl and grown vertically. The seedling phenotypes were documented, and the root lengths were measured 7 to10 days after the salt treatment. For soil experiments, three-week-old plants were watered with NaCl solutions every 3 days (100 ml per pot) with incremental concentrations of 50 mM, 75 mM, and 100 mM NaCl, twice at each concentration. Thereafter, plants were irrigated with regular water until the end of the experiment and plant height and seed yield were recorded.

### Example 1.16. Statistical Analysis

Student's t-test (α=0.05) was performed on measurements such as root length, seed yield, and plant height to compare the performance of WT and transgenic plants. Tukey's method was used for pairwise comparisons among different genotypes (e.g., OsSIZ1-overexpressing, LtRCA-overexpressing, and OsSIZ1/LtRCA co-overexpressing plants) at α=0.05 significance level.

### Example 1.17. Discussion

Based on the aforementioned proof-of-concept experiments, Applicant expects that OsSIZ1/RCA co-overexpressing plants other than Arabidopsis thaliana (e.g., cotton) would produce equivalent or even higher fiber yields under drought and heat stress conditions in laboratory as well as in dryland field conditions. Drought and heat are major environmental stresses that limit cotton production in many regions, such as the Texas High Plains. Therefore, a need exists for the development of drought- and heat-tolerant plant (e.g., cotton) varieties.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A modified plant or seed,
wherein the modified plant or seed comprises:
overexpressed rice SUMO E3 ligase SIZ1 (OsSIZ1), an analog thereof, a homolog thereof, a derivative thereof, or combinations thereof; and
overexpressed Larrea tridentate rubisco activase (LtRCA), an analog thereof, a homolog thereof, a derivative thereof, or combinations thereof.

2. The modified plant or seed of claim 1, wherein the modified plant or seed demonstrates enhanced resistance to environmental stress, and wherein the environmental stress is selected from the group consisting of heat stress defined as temperatures of at least about 10° C. above an optimum temperature for the plant or seed for at least two consecutive days; drought stress defined as lack of water for at least one week; high salt concentrations defined as a soil salt concentration of at least about 50 mM; low temperatures defined as temperatures of at least about 10° C. below an optimum temperature for the plant or seed for at least two consecutive days; and combinations thereof.

3. The modified plant or seed of claim 2, wherein the enhanced resistance to environmental stress is defined by at least one of enhanced crop yield relative to unmodified plants or seeds, enhanced seed yield relative to unmodified plants, display of longer hypocotyl lengths relative to unmodified plants, enhanced growth rates relative to unmodified plants or seeds, enhanced photosynthetic rates relative to unmodified plants or seeds, and combinations thereof.

4. The modified plant or seed of claim 1, wherein the modified plant or seed comprises overexpressed OsSIZ1, wherein the overexpressed OsSIZ1 comprises SEQ ID NO: 1 or a sequence with at least 50% identity with SEQ ID NO: 1.

5. The modified plant or seed of claim 1, wherein the modified plant or seed comprises an overexpressed analog, homolog or derivative of OsSIZ1, wherein the analog, homolog or derivative shares at least 50% identity with SEQ ID NO: 1.

6. The modified plant or seed of claim 1, wherein the modified plant or seed comprises overexpressed LtRCA, wherein the overexpressed LtRCA comprises SEQ ID NO: 2 or a sequence with at least 50% identity with SEQ ID NO: 2.

7. The modified plant or seed of claim 1, wherein the modified plant or seed comprises an overexpressed analog, homolog, or derivative of LtRCA, wherein the analog, homolog or derivative shares at least 50% identity with SEQ ID NO: 2.

8. The modified plant or seed of claim 1, wherein the modified plant or seed is selected from the group consisting of soybean, maize, sorghum, cotton, alfalfa, rice, wheat, barley, potato, legumes, lettuce, tomato, peas, beans, lentils, peanuts, cucumber, hemp, and brachypodium.

9. The modified plant or seed of claim 1, wherein the modified plant or seed comprises a modified seed.

10. The modified plant or seed of claim 1, wherein the modified plant or seed comprises a modified plant.

11. Method of developing a modified plant or seed, said method comprising:
overexpressing rice SUMO E3 ligase SIZ1 (OsSIZ1), an analog thereof, a homolog thereof, a derivative thereof, or combinations thereof in a plant or seed; and
overexpressing Larrea tridentate rubisco activase (LtRCA), an analog thereof, a homolog thereof, a derivative thereof, or combinations thereof, in the plant or seed.

12. The method of claim 11, wherein the over-expressing of each of the genes comprises modifying the expression of at least one endogenous gene in the plant or seed, introducing at least one exogenous gene into the plant or seed, or combinations thereof.

13. The method of claim 11, wherein OsSIZ1 is overexpressed in the plant or seed, and wherein the overexpressed OsSIZ1 comprises SEQ ID NO: 1 or a sequence with at least 50% identity with SEQ ID NO: 1.

14. The method of claim 11, wherein an analog, homolog, or derivative of OsSIZ1 is overexpressed in the plant or seed, wherein the analog, homolog or derivative shares at least 50% identity with OsSIZ1.

15. The method of claim 11, wherein LtRCA is overexpressed in the plant or seed, and wherein the overexpressed LtRCA comprises SEQ ID NO: 2 or a sequence with at least 50% identity with SEQ ID NO: 2.

16. The method of claim 11, wherein an analog, homolog, or derivative of LtRCA is overexpressed in the plant or seed, wherein the analog, homolog or derivative shares at least 50% identity with SEQ ID NO: 2.

17. The method of claim 11, wherein the modified plant or seed is selected from the group consisting of soybean, maize, sorghum, cotton, alfalfa, rice, wheat, barley, potato, legumes, lettuce, tomato, peas, beans, lentils, peanuts, cucumber, hemp, and brachypodium.

18. A method of growing a modified plant or seed in a field, said method comprising:
applying the modified plant or seed to the field, wherein the modified plant or seed comprises:
overexpressed rice SUMO E3 ligase SIZ1 (OsSIZ1), an analog thereof, a homolog thereof, a derivative thereof, or combinations thereof; and
overexpressed Larrea tridentate rubisco activase (LtRCA), an analog thereof, a homolog thereof, a derivative thereof, or combinations thereof.

19. The method of claim 18, wherein the applying comprises applying the modified seed to the field.

20. The method of claim 18, wherein the applying comprises applying the modified plant to the field.


SEQUENCE LISTING (2 sequences)

SEQ ID NO: 1 - DNA, 3393 bp, Oryza sativa
  source 1..3393: mol_type = genomic DNA
aaaaccacaacgaactaccccctcctcgtcgagccgacgcgagagaggaaagtgggttgcggcttgctgcgcgtgtggagtcgccattccccaattcgctgcgccgcccgccggatctcgtcttgccccctgcggcggcggtttgggcccccccttccgatcggtttcccccccgcacatggtcgtggcggcggcggaggtggtggtggtgcgggagtagggaggcgggcgaaccgaggcggcggcgccgatggcggacctggtttccagctgcaaggataaactggcatactttagaataaaggaactcaaagatatattaaatcagctcggcttaccaaagcaaggaaagaagcaggatcttattgatagggtgttggcactcttaacagatgagcaaggtcaaaggcatcatggatggggaaggaaaaattctctcaccaaggaggcagtggcaaaaattgttgatgatacatacaggaaaatgcaaatccaatgtgctcctgatctagccaccaggagccacagcggatcagatttcagtttcaggcctatagaggaagcctatgactctttccagccagaggccaaagttcgctgcatttgcagtagcacaatggttaatgacagcatgatccagtgtgaagatcagcgatgccaagtgtggcaacatttgaattgtgtactcattccagataagcctggggagagcgctgaagttccacccgttttctactgtgaattatgccgactgagtcgggcagacccattttgggtcactgctggaaatccattactcccagtgaaattcgtgtcatctggtgttacaaatgatggaacaagtgtacctcaaagtgtggagaaaagcttccagctttctcgatcagatagagaaactgtccagagacaagaatatgacctccaggtttggtgcatgcttttgaatgacaaagttcagttcaggatgcagtggccccaatatgcagaattgcatgttaatggtatttctgtacgagtagtgactagacctggttctcaattactagggataaatggacgggatgatggtccactgataaccacatgcagtagagagggaattaataaaatttgcttatcaagggtcgatgctcggacattttgctttggtgttcgaattgctaaacggaggactgttgctcaggttttgaacttagttccaaaggaagctgaaggagagtcttttgagcatgctcttgctcgtgttcggcgctgtctcggaggtggagacactgcagagaatgctgatagtgacagtgatttggaagtggttgcggagtctgttacagtcaaccttcgttgccctaatagtggatccagaatgaggattgctgggagattcaagccttgcattcacatgggttgttttgatcttgaaactttcgtggaattgaatcaacggtcccgcaagtggcaatgtccaatatgtttaaagaattactctcttgagagcttgatgattgatccttacttcaataggattacttctctgttgcgcaattgcaatgaggatgtcaatgaggttgatgttaagcctgacggatcttggcgtgtgaagggtgatgctgcaagtagagaattatctcagtggcatatgcctgatggtaccctttgtaatcctaaggaagatgtcaaacctgccatgcaaaatggaaatgaacaaatgatggaaggtacttctgatggacagaaatctttgaaaattggaataaagagaaatccaaatggaatctgggaagttagtagcaaagcagatgacaagaagccttctgtggttggaaatcgcatgcaaaacaatagtgggttccgagctctaaacaacattatgcatatgagcaatagcccaactagtagttatagagacggggaagacccaagtgtgaaccaagagagcaataggcatgttgacttatcattgaacaatggtaataatgagtttgacagtttctctctcaattttggccaagcatgcaatacagatgatagaccacagcaacaacataatgccacagacgtcattgttcttagtgattctgatgaagagaatgatgctatggtttgtccaccagctgtctatgacaatactaccactgcaaatggtagtggttttcctttcaccactaatggtattggatatactgaaaggtaccaggaagatgccggcgttggtacaagtggccttggtttattgagtaacaatgttgatgattttgagatgaataactggcaaatgcattcttcttatcaacaacctgaacaaggcttccagttttttgggaatgatactgatgtccataatacttttgttggttcacacaattcctttggcttagcaccaaatgactattctcttgattgtaatgttggcgtagaggaggcttcggtaactcctgctctttcagtctgccggaatagtaatgaaatgcatggaagtttggttgataacccactggctttggttggcgatgatccatccttgcaaatttttcttccaagtcaaccttcctctgttcctcttcaggaagaacttagcgagcgtgctaatgcaccaaatggggttcagtctgatgattggatatcccttacactcgcagcgggtggaggtggtaacgaagagcctgcacctgctgatgtcaattcacagccacaaattccatcaacagagacagggatcgaaccattgaccgatgctgcttctgcatttctgagcacaaacattgaaagacgtagcggagctgatttaaatccaagaaggatagaaaatatattttctcatcctcgccagcctcggtctgttaggcctcgactctgtttatcaatagatactgattctgagtagtttggacatcataacggggtaactgagtttgcattagtttggcaaagctgccatccagaaatcatgatttatactgaggtggggttatcggtcgtctggttgatgtaaagaaaaacaccagcattgatgctttgttgcctcaatggtttacaagctttcaagtgttctatttgatccaggagggatctgcatagaggacatctgatggttggtatagaaaattttctaactgggtatcgaggcttaatgtggcgactggagcagtttgtacattttttttgttttgacttttactggatataaggaatagaggtggggtcatccctggaccctggatgcaagacaaatacatgtgaatgttttgggtgtaccatcattgtatttagggtttgggggtactaatttagttgtttattaacctggtatttgatggagaataaattattcctggaagtggcggccaataaacagagtcgttggggttttacttgggtaaaaaaaaaaaaaaaaaaacc

SEQ ID NO: 2 - DNA, 1431 bp, Larrea tridentata
  source 1..1431: mol_type = genomic DNA
atggctgctgcctactccaccgtcggagctgtcaacagggcaccgctgagcttgaatggntctggtgcgagagcttcattggttccaagcactgccttcttcggcagcagcttgaagaaatctgctgcgaaattccccaaggcgtcatcaggaaacttcaagattgttgcacaagaaattagtgaggatcagcagaccgacaaggacaaatggaagggtcttgcctatgacatttctgatgatcaacaggatatcactagaggaaagggtatggttgatactctcttccaagctcccatgcaatctggcactcactatgctgtcatgagttcttacgactacatcagtcaaggacttcgccagtacaacttggacaacaacatggacggtttctacatagcaccagccttcatggacaagcttgtcgtccacatcaccaagaacttcttgagtctccctaacatcaagattcctctgatcttgggtatctggggaggcaaaggtcaaggaaaatctttccaatgtgaacttgtttttgccaagatgggaatcaaccccatcatgatgagtgctggagagctggaaagtggaaatgcaggagagcccgcaaagctgatcaggcaaaggtatcgtgaagcagctgatatcatcaagaagggaaaaatgtgctgcctgtttatcaacgatctcgatgccggagctggtcgtatgggtggaacaactcaatacactgttaacaatcagatggtgaatgccaccctcatgaacatagctgacaacccaacaaatgtccagcttcctggcatgtacaacaaggaagagaaccctcgtgttcccatcattgtcactggtaacgacttctcaacattgtatgcccctcttatccgtgatggccgtatggagaaattctactgggcacccaccagggaggaccgtattggtgtctgcaaaggaattttcagaactgacaacgttgctgacgatgatattgtcaagcttgttgacactttccctggccaatccattgatttcttcggtgccctgagggcaagagtttaccatgatgaagtgaggaagtgggtttccgaagtcggtgtcgacactattggcaagaagctggtcaactcaaaagaaggacccccttcatttgagcagcccaagatgacaattgataagctccttggctacggaggcatgcttgtccaggagcaggagaacgtcaagagagttcaattagctgacaaatacatgtccgaggctgctcttggtgatgctaataacgatgctattaaaagaggaactttctacgggggccaagccgcccagcaagtgggtaatgttcctgttcctgaaggttgtactgatcctcaggcaacgaactacgatccaacagctaggagtgacgatggaagctgtgtgtacaagttttaa